(19) United States
(12) Patent Application Publication
(10) Pub. No.: US 2017/0181756 A1
(43) Pub. Date: Jun. 29, 2017

(54) LIMITED USE ACETABULAR REAMER

(71) Applicant: Biomet Manufacturing, LLC, Warsaw, IN (US)

(72) Inventors: William Jason Slone, Silver Lake, IN (US); Matthew D. Munsterman, Warsaw, IN (US)

(21) Appl. No.: 15/399,945

(22) Filed: Jan. 6, 2017

Related U.S. Application Data
(63) Continuation of application No. 13/761,255, filed on Feb. 7, 2013.
(60) Provisional application No. 61/595,860, filed on Feb. 7, 2012.

(51) Int. Cl. A61B 17/16

(52) U.S. Cl. CPC A61B 17/1666; A61B 17/1631; A61B 2017/00964

(57) ABSTRACT

A limited use acetabular reamer can be used to perform selected procedures. The limited use reamer can be formed of low cost materials and be assembled in a limited number of steps. For example, a cutting portion can be formed or a metal and a body portion can be formed of a polymer.

### CROSS-REFERENCE TO RELATED APPLICATIONS

This application claims the benefit of U.S. Provisional Application No. 61/595,860 filed on Feb. 7, 2012. The entire disclosure of the above application is incorporated herein by reference.

### FIELD

The subject disclosure relates to instrumentation, and particularly to instrumentation that can be limited or single use for performing a surgical procedure on a subject.

### BACKGROUND

This section provides background information related to the present disclosure which is not necessarily prior art.

During a surgical procedure on a patient, various surgical instruments can be used. A surgical procedure can include removing selected portions, including portions of a joint. For example, during a procedure on a patient, a hip can be replaced, or at least a portion thereof, requiring removal of a portion of a subject's natural acetabulum. A surgical instrument can be used to remove a portion of the natural acetabulum, which can be referred to as an acetabulum reamer.

### SUMMARY

This section provides a general summary of the disclosure, and is not a comprehensive disclosure of its full scope or all of its features.

A surgical procedure can be performed on a patient. In performing the surgical procedure, it can be selected to use a surgical instrument. Surgical instruments can include single use or limited life instruments to assist in minimizing cross-contamination from subject to subject.

In a surgical procedure, a tool, such as a driver, can be used to drive a reaming instrument. The tool or the driving portion can be positioned exterior to the patient and generally have limited access to and/or ability to contaminate the patient. Although it is understood that the power tool portion can be sterilized or cleaned and can be used within a sterile field or clean field, a selected portion of the instrumentation is positioned within the patient and substantially within the sterile field. Accordingly, including portions of the instrumentation that can be provided as pre-sterilized and for a single or limited use can minimize or eliminate the possibility of patient to patient contamination or infection.

A single use instrument or instrument portion can include an acetabulum reamer. The single use acetabular reamer can be provided to include low cost materials to enhance the ability for a single use and minimize cost. A single use acetabular reamer can include an efficiently or quickly produced body, such as a body formed of a polymer that includes or is coupled to a metallic reaming blade. The acetabular reamer body can then be used for a single patient and discarded without significant cost. The single use or limited use acetabular reamer need not be cleaned or sterilized between procedures to maintain a low or selected procedure cost.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a view of a power tool and an acetabular reamer;

FIG. 2 is detailed view of the reamer portion of the instrumentation;

FIG. 3 is a perspective view of an acetabular reamer according to various embodiments;

FIG. 4 is a perspective view of an acetabular reamer according to various embodiments;

FIG. 5 is a detailed view of an acetabular reamer according to various embodiments;

FIG. 6 is a perspective view of a reamer according to various embodiments;

FIG. 7 is an environmental view of an acetabular reamer instrumentation in use;

FIG. 8A is a plan view of a mold including cutting blades;

FIG. 8B is a plan view of a closed mold; and

FIG. 8C is a plan view of a completed acetabular reamer in a mold portion.

Corresponding reference numerals indicate corresponding parts throughout the several views of the drawings.

### DETAILED DESCRIPTION

Example embodiments will now be described more fully with reference to the accompanying drawings.

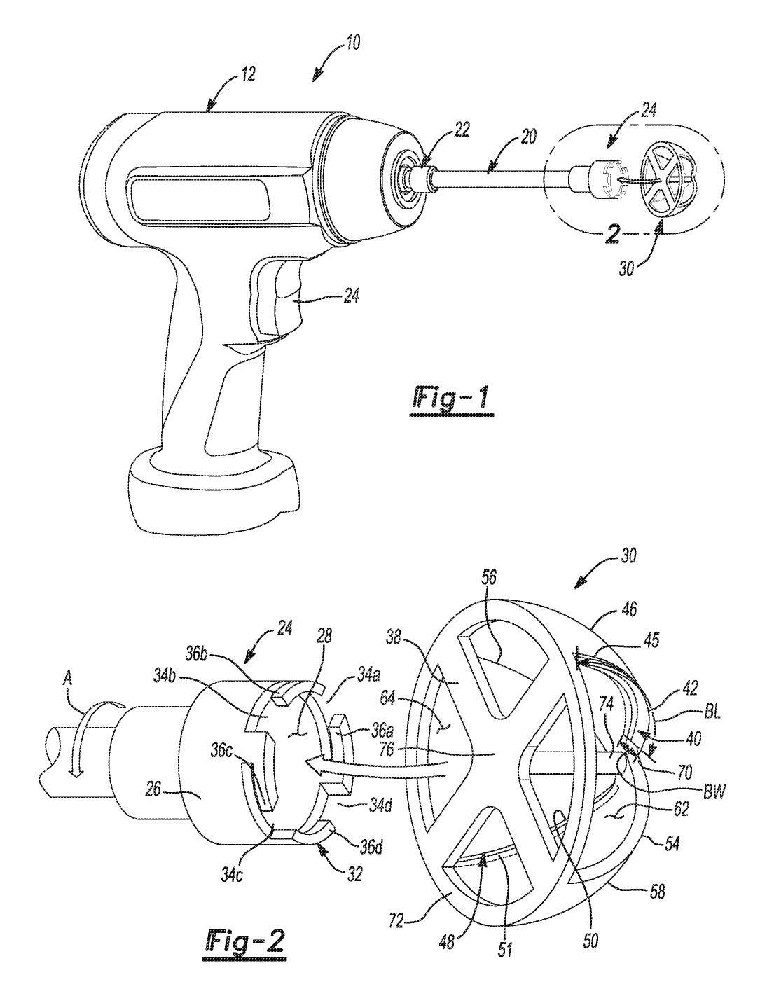

An instrument assembly 10 is illustrated in FIG. 1. The instrument assembly 10 can include a power drill 12 that can be provided according to various embodiments, such as a power drill and/or the Coughlin Small Joint Reamer System, sold by Stryker Corporation. The power drill or drill motor 12 can be electrically, hydraulically, or pneumatically or otherwise powered. A trigger portion 14 can be provided to allow a user, such as a surgeon, to selectively power and depower the motor 12 to rotate or spin a driver arm 20. The driver arm 20 can be a driver arm similar to a straight APEX driver sold by Symmetry Medical Inc. Generally, the driver arm 20 can include a motor coupling portion 22 that can couple with a chuck assembly or other appropriate assembly with the drill motor 12. At a distal or reaming engaging end 24, can be a reamer head engagement portion.

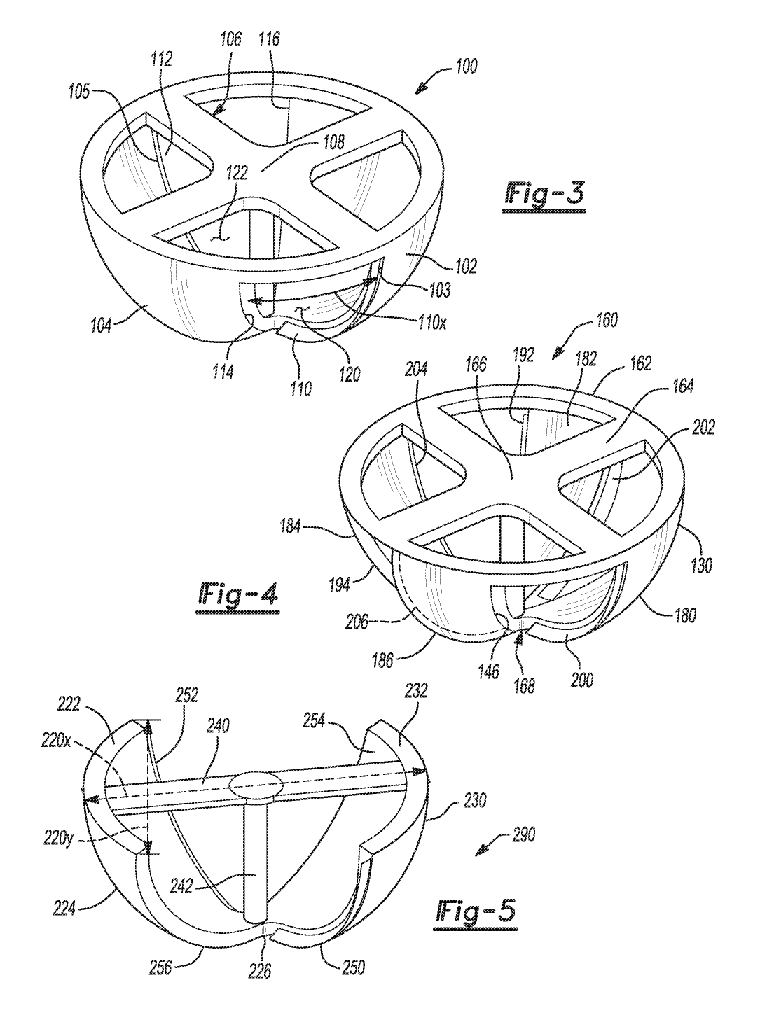

With additional reference to FIG. 2, the reamer head engagement portion 24 can include a solid or hollow member 26. If the member 26 is hollow an open or gathering area 28 can be provided to assist in holding reaming portions or reamed material formed with a reamer 30. The body portion 26 can include a rim or distal end 32 that can include one or more bayonet slots 34a-34d that allows for engagement and disengagement of the reamer body 30. As is generally understood by one skilled in the art, the reamer 30 can include one or more cross members or driver arm engagement portions 38 that can pass through the bayonet slots 34a-34d and swivel into the grasping arms and be held in position by retaining fingers 36a-36d. The retaining fingers 36a-36d can hold the reamer 30 in place during rotation of the driver arm 20 in a direction, such as a direction of rotation arrow A. During rotation in the direct of arrow A, a reaming blade 40 having a leading or cutting edge 42 to engage and ream a portion of a subject (e.g. an acetabulum of a patient as illustrated in FIG. 7).

The acetabular reamer 30 can include the blade 40 that is coupled to or fit within a leading edge 45 of a wall 46 (where the wall 46 can be formed of a polymer material) according to various formation techniques, including those discussed herein. The blade leading edge 42 can define a leading edge of the reamer 30 as it rotates in the direction of arrow A driven by the driver arm 20. The reamer 30 can also include a second blade 48 having a second blade leading edge 50 that is placed in a second leading edge 51 of a second wall portion 58. The two blades 40, 48 allow for at least two cutting blades to be formed on the reamer 30. Both of the wall portions 46, 58 and both of the blades 40. 48 can be curved or formed on arcs to define at least a portion of a sphere, such as a hemisphere.

Each of the blades 40, 48 can be formed in appropriate dimensions. For example, the blades 40, 48 (or according to any appropriate embodiment) can have a length BL that is at least as greater as a width BW, but is generally longer than its width. For example, the blades 40, 48 can be about 5 times to about 10 times longer than their respective widths. In a particular example, the blade 40 can include a blade length BL of about 5 centimeters (cm) and a blade width BW of about 1 cm. In addition, the blades 40, 48 can be provided at differing lengths, as discussed herein.

Between each of the leading edges or cutting edges 42, 50 can be respective trailing wall edges 54 and 56. The trailing wall edges 54, 56 can be formed at edges of wall portions 46, 58 opposite or trailing the blades 40, 48. Openings 62 and 64 can be defined between the leading edge 42, 50 of the blades 40, 48 and the trailing edges 54, 56. Reaming material can therefore be held within the reamer body 30 during a reaming procedure and assist in removal of the reaming material (e.g. bone reaming or chip portions) periodically or at an end of a reaming procedure.

The two wall portions 56, 58 can be substantially arcuate and extend from or towards an apex 70. The walls can extend from the apex 70 to a rim or edge 72. Also, a reinforcement or member bar 74 can extend from the apex 70 to an intersection 76 of the engagement bars 38. The engagement bars 38, either alone or in combination with the member 74, can transfer a force from the tool to the blades 40, 48 through the walls 46, 58. The reinforcement member 74 can extend substantially linearly from an apex 70 to the intersection 76 and can extend along an axis of rotation of the reamer 30. The engagement bars 38 can extend in a substantially cruciform fashion or configuration between opposing points on the edge or rim 72 of the reamer body 30. The engagement bars 38, as discussed above, can then pass through the slots 34a-34d and be rotated to be engaged by the arms 36a-36d.

The portions of the reamer 30 are discussed above. The various portions of the reamer 30 can be formed of different materials. The portions of the reamer body not including the blades 40, 48 can be formed of a polymer material or fiber reinforced polymer material. The selected material can be low cost and easy to form. The body of the reamer 30, generally the portion of the reamer 30 not including the blades 40, 48, can be formed according to various procedures, including those discussed herein. The blades 40, 48 can be formed of an appropriate material, such as titanium or titanium alloys, steel or steel alloys, or other appropriate metals or materials that can be sharpened or honed to appropriate cutting edges 42, 50. Accordingly, the reamer 30 can be formed of two materials that can include be substantially low cost and formed of a quickly produced polymer body and a selected rigid or edge holding material for the blades 40, 48. It is understood, however, that a single use reamer may also be formed of a single material. For example, a reinforced polymer material that can maintain a reaming edge for a limited time or a biocompatible, but inexpensive, metal or metal alloy. Thus, the reamer 30 need not be formed of two materials.

With additional reference to FIG. 3, an acetabular reamer 100 is illustrated. The acetabular reamer 100 can include portions similar to the acetabular reamer 30 discussed above, and will not be discussed in detail here. For reference, however, the acetabular reamer 100 can include arcuate walls 102 and 104 that can define at least a portion of a sphere, similar to the walls 46, 58 discussed above. The reamer 100 can further include a driver connecting or engagement portion 106 intersecting an intersecting portion 108 similar to the connecting portion 38 discussed above. The arcuate walls 102 and 104 can include leading edges 103, 105 that have respective blades 110 and 112 connected therewith. Accordingly, each of the walls 102 and 104 can also include trailing or following edges 114 and 116, respectively. The reamer 100 can include the blades 110, 112 that are formed of selected material, such as a metal, that can be different from the portions of the reamer 100 formed of a different material, such as a molded polymer, as discussed above.

Spaces between respective blade 110 and trailing edge wall 114 and the other blade 112 and wall trailing edge 116 can define opening or spaces 120 and 122. The spaces 120 and 122 can be similar to the spaces or openings 62, 64 of the acetabular reamer 30 discussed above. As discussed above, material can move into an opening or central region defined by the walls 102, 104 of the reamer 100. The openings 120, 122, however, can be at a dimension that is smaller than the similar dimension of the openings 62, 64 of the reamer 30. The dimension can include a spatial dimension on a surface of the curved surface defined by the wall 102 between the leading edge of the blade 110 and the trailing wall edge 114 and generally be a dimension 110x. The dimension 110x can be any appropriate dimension, but be different from the dimension of the opening 62, 64 discussed above. Accordingly, it will be understood by one skilled in the art that the dimension 110x between the blade edge 110 and the wall trailing edge 114 can be selected for various purposes. For example, a smaller dimension or smaller opening can allow for easier collection and greater collection of reamed material within the reamer 100. Accordingly, the opening size 110x can be selected based upon a selected amount of material to collect within the reamer 100, or reamer according to any appropriate embodiment.

With reference to FIG. 4, an acetabular reamer 160 is illustrated. The acetabular reamer 160 can include portions that are similar to the portions of the acetabular reamers 30, 100 discussed above, and will not be discussed in detail here. For example, the acetabular reamer 160 can include an upper rim or edge 162 which is interconnected from which extends a connection portion 164 having an intersection 166 for connecting to the instrument body 26. Extending from the upper edge 162 towards an apex or center region 168 are a plurality of arcuate walls 180, 182, 184 and 186. Each of the walls can include a trailing or following edge or end, respectively 190, 192, 194, and 196. Each of the trailing edges 190-196 can be similar to the trailing edges discussed above, such as the trailing edges 54, 56. The trailing edges 190-196 are opposite to or face leading edges of walls in which are placed cutting or reaming blades 200, 202, 204 and 206. Each of the blades 200-206, therefore, can define leading edges of the reamer 160. Each of the blades 200-206 can be formed of appropriate material, as discussed above, for reaming selected portions, such as a bone of an anatomy.

The reamer 160 can be formed similar to the reamers 30, 100 discussed above, except that the reamer 160 can include four blades 200-206. Each of the four blades can be formed on an arc that extends from near the edge 162 towards the apex 168. Accordingly, the walls 180-186 and the blades 200-206 can together define at least a portion of a sphere, such as a hemisphere. Also, each of the blades 200-206 can be formed to ream material of a selected subject. Having a plurality of blades, such as more than two, can reduce the amount that each of the blades would need ream to allow for a selected reaming speed. Additionally, a size and/or pitch of each of the blades can be reduced by including a plurality of blades. It will be understood, however, that any appropriate number of blades can be provided in the reamer 160, or any of the reamers according to various embodiments. Providing four, or more or less, blades, can be selected for achieving a selected amount of bone removal, an aggressiveness of the reamer, and other appropriate features. Nevertheless, the reamer 160 can be formed similar to the reamers, such as the reamers 30, 100, discussed above, including the blades 200-206 formed of a selected material (e.g. such as a metal or metal alloy) and the other body portion of the reamer 160 can be formed of a selected different material, such as a moldable polymer.

With reference to FIG. 5, a reamer 220 is illustrated. The reamer 220 can include portions that are similar to portions of the reamers illustrated above, such as an upper edge 222 and an arcuate wall 224 that extends towards an apex 226 from the upper edge 222. A second wall 230 can also extend from an upper edge 232 towards the apex 226. The edges 222 and 232 can define arcs of a circle that can be substantially continuous if imaginary lines were to connect the two edge portions 222, 232. Nevertheless, as illustrated in FIG. 5, the arcuate wall portions 224 and 230 are disconnected at the edge portions such that the first edge 222 is not directly connected to the edge 232 at the upper edge region on the circle defined by the edge portions 222, 232 .

Although the two edges 222 and 232 are not interconnected on a circle, a linear or other connection member 240 can extend between the two edge portions 222 and 232 for connection to the drive portion 26, as discussed above. A support member or rod 242 can extend from the connection member 240 towards and/or connect to the apex 246.

As illustrated in FIG. 5, the acetabular reamer 220 can include substantially two independent wall portions 224 and 230 that are interconnected at the apex 226 and the edge portions interconnected with the connection member 240. The acetabular reamer 220, therefore, can include blades 250 and 252 positioned in leading edges of the walls 224, 230 and trailing wall edges 254 and 256. The acetabular reamer 220, therefore, can include a first dimension 220x along the sides, such as substantially along the dimension of the connection member 240, that is greater than a second dimension 220y substantially transverse to the first dimension 220x. Generally, the long or greater dimension 220x can extend along the connector 240. The second dimension 220y can be transverse to the dimension 220x and generally to be defined as a greatest dimension from an outer edge or wall portion of the wall portion 220 by the blade 252 to an outer edge portion of the wall portion 224 by the following edge 256. It will be understood that the second wall portion 230 can include a similar or different dimension. Nevertheless, the two dimensions 220x and 220y that are different can form a substantially narrow or small clearance dimension for inserting the acetabular reamer 220 into a selected anatomical portion. Accordingly, the dimension 220y can allow the acetabular reamer 220 to be used in a less invasive manner than substantially cylindrical or hemispherical reamer. Nevertheless, the acetabular reamer 220 can be formed in the manner and of materials substantially similar to the reamers discussed above, including the blades 250 and 252 formed of a selected material, such as a metal or metal alloy and the remainder portion of the reamer formed of a second material, such as a moldable polymer.

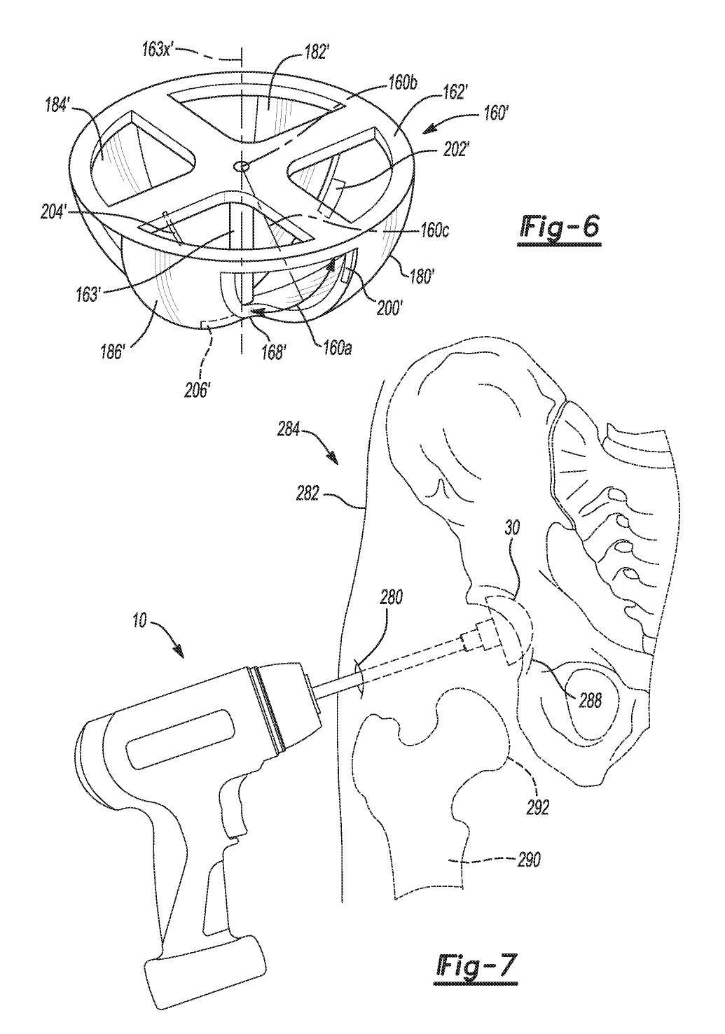

With reference to FIG. 6, an acetabular reamer 160′ is illustrated. The acetabular reamer 160′ can include portions that are similar to the acetabular reamer 160 illustrated in FIG. 4 and discussed above. The acetabular reamer 160′, however, can also include portions that are similar to the acetabular reamer 30, 100, and 290, or according to various embodiments. Regardless, the following discussion generally relates to the differences of the acetabular reamer 160′ illustrated in FIG. 6 that differ from the reamer 160.

As discussed above, the acetabular reamer 160′ can include portions similar to the acetabular reamer 160 which will include reference numerals similar to that in FIG. 4 augmented with a prime. Accordingly, the acetabular reamer 160′ can include an upper annular rim 162′ and an apex 168′ with four walls 180′, 182′, 184′, and 186′ extending along arcs from the upper rim 162′ towards the apex 168′. Each of the walls 180′-186′ can define leading and trailing edges, as discussed above. Each of the leading edges of the walls can include a blade member. A first blade member 200′ can be positioned in the first wall 180′, a second blade member 202′ positioned in a second wall 182′, a third blade member 204′ positioned in a third wall 184′, and a fourth blade member 206′ positioned in a fourth wall 186′. Each of the blade members 200′-206′, however, do not extend along an entire arc from the upper rim 162′ to the apex 168′.

As illustrated in FIG. 6, the reamer 160′ can define a portion of a sphere. An arc 160a of the sphere can be defined from a center 160b, which can be a center of the sphere of the reamer 160′ and a radius 160c. The arc 160a extends from the upper rim 162′ to the apex 168′. The arc 160a can also be substantially equivalent to an arc defined by the walls 180′-186′ or at least the leading edges thereof. The blade members 200′-206′ may only define at least a portion of the arc 160a. For example, the first blade member 200′ can define about 25% of the arc 160a. Each of the other respective blade members 202′-206′ can also each respectively define another 25% of the arc 160a. Each of the blade members 200′-206′ can also be positioned at differing locations along the length of the arc 160a in the respective leading edges of the walls 180′-186′.

As illustrated in FIG. 6, the first blade member 200′ can be positioned substantially near or adjacent to the upper rim portion 162′ while the fourth blade member 206′ can be positioned near or substantially adjacent to the apex 168′. The two other blade members 202′ and 204′ can be positioned at locations intermediate the first blade member 200′ and the fourth blade member 206′. It will be understood that none of the blade members need engage or contact either the upper rim 162′ or the apex 168′, but can be positioned near them according to the specific design of the acetabular reamer 160′.

It will be further understood that the blade members 200′-206′ need not together define the entire arc 160a. The various blade members 200′-206′ can be positioned at any location on the various walls 180′-186′ on the leading edges thereof to define all or a portion of the arc 160a together. It is understood in operation that the acetabular reamer 160′ generally spins or can be rotated in a direction such that the blade members 200′-206′ or a leading edge engages a portion, such as an acetabulum, as exemplary illustrated in FIG. 7 below. Accordingly, during rotation, each of the blade members 200′-206′ can ream or remove material in a annular or circular region extending around a central axis 163′x, such as one defined through the centerpoint 160a and also generally along the support post 163′. The axis 163′x can extend through the post 163′ around which the blade members 200′-206′ rotate. Accordingly, each of the blade members can define a selected region of resection that can be substantially distinguishable and separate from resection regions of the other blade members. It will be understood, however, that the blade members 200′-206′ can also be positioned on the acetabular reamer 160′ to partially or substantially overlap, such as overlap by 5-10% or entirely overlap for multiple blade members to ream the same area. Nevertheless, each blade can define up to 100%, including about 5% to about 99%, and further including about 10% to about 90%, of the complete arc.

Providing a plurality of the blade members 200′-206′ that do not or do not substantially overlap or only remove or resect a portion of the length of the arc 160a can be provided for various reasons. For example, reducing the amount of torque that a user, such as a surgeon, may feel with single large blades, or adjusting the aggressiveness of the reamer to give the surgeon more precise control of a reaming depth. Additional examples include reducing friction or interaction at each of the leading edges of the walls 180′-186′ to reduce forces applied to each of the walls 180′-186′. Additionally, an initial rotation of the acetabular reamer 160′ can be reduced or forces or stresses applied to the reamer 160′ can be reduced. Accordingly, the main body portion of the reamer 160 can be formed of selectively weak materials or formed as a thin member if the body portion does not need to resist forces that may be applied to the reamer 160′ if each of the blade members 200′-206′ were formed the entire length of the arc 160a. Thus, the reamer 160′ can be formed of less material or selected less expensive materials to reduce costs and manufacturing time of the reamer 160′. It will be understood that the acetabular reamer 160′, however, can be formed according to various embodiments as discussed.

With reference to FIG. 7, the acetabular reamers according to various embodiments, including those discussed above, can be used in any appropriate manner, such as in a manner understood by those skilled in the art. As illustrated in FIG. 7, and described herein briefly, the acetabular reamer 30, illustrated above, is interconnected with the drill assembly 10. The acetabular reamer 30 can be passed through an incision 280 formed in a soft tissue, such as a dermis 282 of a patient 284. It will be understood that the patient 284 can be any appropriate patient, such as a human patient, an animal patient, or other appropriate patient. The patient 284 can include boney structures, such as a pelvis 286 that defines or includes an acetabulum 288. During reaming, a femur 290 can be dislocated such that a head portion 292 of the femur 290 is not seated in the acetabulum 288 during the reaming procedure. The reamer 30 can be used according to a conventional known manner to ream the acetabulum 288 of the patient 294 for a selected procedure. Subsequent to or after a selected amount of reaming the acetabular reamer 30 can be discarded in a substantially appropriate manner. For example, the acetabular reamer 30 can be discarded in a sharps container or other manner to ensure that the reamer 30, after use on the patient 284, is not used on a second or subsequent patient. Accordingly, the limited use acetabular reamer, according to various embodiments, can be used for a selected single procedure and then discarded.

The limited use acetabular reamers can ensure that the cutting edges of the blades remain substantially sharp for the procedure and are not dulled by prior procedures or need to be checked for sharpness. Additionally. cross-contamination from one patient to another can be substantially eliminated by using the reamer 30 on substantially only the single patient 284 during a single procedure. Thus, cross-contamination from one patient to another and communication of diseases can be substantially eliminated by possible inefficient cleaning and sterilization of a multiple use reamer.

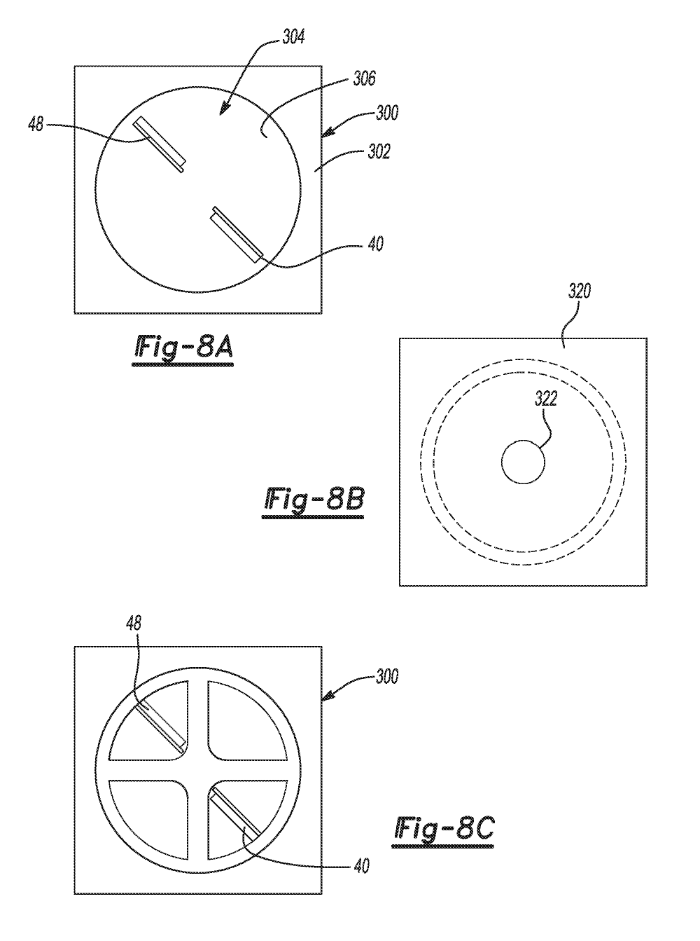

Discussed above are limited use acetabular reamers being shaped according to various embodiments. It will be understood, however, that the acetabular reamers can be formed of two materials, such as blades or cutting edges being formed of a first material having selected properties (e.g. a metal having a substantially stiff or ridged physical property for allowing efficient cutting of a subject and a body portion formed of an inexpensive and/or easily produced and formed material.) According to various embodiments, as illustrated in FIGS. 8A-8C, a method of forming the acetabular reamer 30 is illustrated.

With initial reference to FIG. 8A, a mold 300 can include a mold body 302 and a mold cavity 304 and can be used for forming the reamer 30. The mold cavity 304 can include a surface 306 that can be used to define an external dimension of the reamer walls 46, 58. Accordingly, the surface 306 of the cavity 304 can substantially define a portion or all of a hemisphere that can be filled with a moldable material during a molding process.

Positioned within the cavity 304 prior to molding or positioning a moldable material into the cavity 304 can be the blade members 40 and 48. The blade members 40 and 48 can be positioned within the cavity 304 near the surface 306 in any appropriate manner, such as on investment portions which are portions that can be removed after at least a portion of the molding procedure. The blades 40, 48 can also otherwise be held within the cavity 304.

Once the blades 40, 48 are positioned within the cavity 304 of the mold 300, a top mold body or portion 320 can be positioned on the mold body 300, as illustrated in FIG. 8B. The top mold portion 320 can include a structure that fits within a portion of a cavity 304 to define a space between the top portion 320 and the surface 306 of the cavity 304 that defines a thickness of the walls 46 and 48 of the reamer 30 and the support portion 74 extending from the apex 70 of the reamer 30. An opening 322 in the top mold portion 320 can allow a material to be injected into the mold cavity 304 through the top mold portion 320. After a selected time, the top mold portion 320 can be removed to allow access to the cavity 304, as illustrated in FIG. 8C.

The cavity 304 can then include at least a portion of the molded body of the reamer 30 with the blades 40, 48 fixed within the body. The support portion 74 can also be extending from the apex 70. A separately formed connection portion 38 can then be fixed to the body of the acetabular reamer to finish the acetabular reamer 30. The connection 38 portion can be connected to the other portions of the reamer body by any appropriate means, such as epoxy adhesives, welding, or other appropriate techniques.

Accordingly, the acetabular reamer 30 can be formed by molding a material, such as an appropriate polymer material, around blades 40, 48 that are formed separately. The process can be one that is limited in a number of steps due to allowing prefinishing of the blades 40, 48 prior to positioning the blades 40, 48 within the molding cavity 304 and a substantially single step injection molding and second step connection of fixing the connection portion 38 to the acetabular reamer.

In addition, the materials for forming a majority of the acceptable reamer 30, or according to any of the various embodiments, can be a selected low cost material, such as selected polymers. Nevertheless, the polymers can be selected to include appropriate physical structures or characteristics to allow for driving them with the drill motor 12 to ream a selected portion of the anatomy. Additionally, the reamer body, according to various embodiments, can include an appropriate stiffness and toughness to ream the selected portions of the anatomy.

It will be understood, however, that the blades 40, 48 can be positioned with the reamer according to various appropriate embodiments alternatively to that illustrated in FIGS. 8A-8C. For example, the blades 40, 48 of the reamer 30 can be press fit into a polymer body to form the reamer 30. Also, the blades 40, 48 can otherwise be connected to a body of the reamer 30, such as by adhesion to a portion of the wall to allow the blades to engage the anatomy during reaming. For example, the blades can be adhered to an exterior portion of the walls.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A single use acetabular reamer, comprising:
a body including a wall extending from an apex to a rim edge, the body formed of a disposable material: such that the single use acetabular reamer is configured to be discarded after a single use: and
a blade member having a length greater than a width and formed of a first metal, wherein the blade member includes:
a sharpened leading edge operable to engage a surface of a subject: and
a trailing edge fixed in a leading edge of the wall.

2. The single use acetabular reamer of claim 1, wherein the wall has an exterior surface that extends along an arc from the apex to the rim edge and the wall defines at least a portion of a sphere.

3. The single use acetabular reamer of claim 1, wherein the disposable material comprises a second metal, wherein the first metal is harder than the second metal.

4. The single use acetabular reamer of claim 1, wherein the disposable material comprises the first metal.

5. The single use acetabular reamer of claim 2, wherein the blade member defines at least a portion of the sphere.

6. The single use acetabular reamer of claim 1, wherein the blade member includes a first blade member and a second blade member both having a length greater than a width and formed of the first metal;
wherein the wall includes a first wall and a second wall both extending from the apex to the rim edge;
wherein the first blade member is fixed within a first leading edge of the first wall and the second blade member is fixed within a second leading edge of the second wall and both the first blade member and the second blade member are operable to engage the surface of the subject.

7. The single use acetabular reamer of claim 6, wherein the first wall, the first blade member, the second wall, and the second blade member all define at least a portion of a sphere.

8. The single use acetabular reamer of claim 7, further comprising:
a cruciform driver connection member extending between the first wall and the second wall at the rim edge and operable to engage a drive shaft; and
a support member engaging and extending from an intersection of the cruciform driver connection member to the apex;
wherein the rim edge is substantially annular;
wherein the cruciform driver connection member either alone or with the support member is operable to transfer a force from the drive shaft to the first blade member and the second blade member.

9. A single use acetabular reamer, comprising:
a first blade member having a length greater than a width and formed of a first disposable metal, such that the single use acetabular reamer is configured to be discarded after a single use;
a first wall extending along a first arc from an apex to an upper edge and defining a leading edge and a trailing edge;
a driver connection member extending from the upper edge; and
a support member extending from near the apex towards a plane defined by the upper edge and engaging the driver connection member;
wherein the blade member is fixed within the leading edge of the first wall and operable to engage a surface of a subject;
wherein the blade member extends along a substantial portion of a length of the first leading edge from near the apex to near the upper edge.

10. The single use acetabular reamer of claim 9, further comprising:
a second wall extending along a second arc from the apex to the upper edge defining a second leading edge and a second trailing edge;
a second blade member having a length greater than a width and formed of the first disposable metal;
wherein the first wall and the second wall together define a portion of a sphere.

11. The single use acetabular reamer of claim 10, wherein the second blade member extends along a substantial portion of a length of the second leading edge from near the apex to near the upper edge.

12. The single use acetabular reamer body of claim 11, wherein the first blade member and the second blade member define a portion of the sphere.

13. The single use acetabular reamer of claim 1, wherein the first disposable metal is a metal alloy.

14. The single use acetabular reamer of claim 1, wherein the first wall and the driver are formed of the first disposable metal.

15. The single use acetabular reamer of claim 1, wherein the first wall and the driver are formed of a second disposable metal.

16. The single use acetabular reamer of claim 15, wherein the first disposable metal is harder than the second disposable metal.

17. A single use acetabular reamer comprising:
a first wall extending along a first arc from an apex to an upper edge and defining a first leading edge and a first trailing edge;
a first arcuate blade including a length extending along at least a portion of the first wall, wherein the length of the first arcuate blade is greater than a width of the first arcuate blade, wherein the first arcuate blade further includes a first sharpened leading edge configured to engage a surface of a subject, wherein the first arcuate blade includes a first blade trailing edge embedded within the first leading edge of the first wall;
a second wall extending along a second arc from the apex to the upper edge and defining a second leading edge and a second trailing edge; and
a second arcuate blade including a length extending along at least a portion of the second wall, wherein the length of the second arcuate blade is greater than a width of the second arcuate blade, wherein the second arcuate blade further includes a second sharpened leading edge configured to engage a surface of a subject, wherein the second arcuate blade includes a second blade trailing edge embedded within the second leading edge of the second wall;
wherein the first and second walls and the first and second arcuate blades comprise disposable metal, such that the single use acetabular reamer is configured to be discarded after a single use.

18. The single use acetabular reamer of claim 17, wherein the first arcuate blade extends along a substantial portion of a length of the first leading edge from near the apex to near the upper edge.

19. The single use acetabular reamer of claim 17, further comprising:
a third wall extending along a third arc from an apex to an upper edge and defining a third leading edge and a third trailing edge; and
a third arcuate blade including a length extending along at least a portion of the third wall, wherein the length of the third arcuate blade is greater than a width of the third arcuate blade, wherein the third arcuate blade further includes a third sharpened leading edge configured to engage a surface of a subject, wherein the third arcuate blade includes a third blade trailing edge embedded within the third leading edge of the third wall.

20. The single use acetabular reamer of claim 17, further comprising:
a driver connection member extending from the upper edge; and
a support member extending from near the apex toward a plane defined by the upper edge and engaging the driver connection member.
